(19) United States
(12) Patent Application Publication
(10) Pub. No.: US 2011/0153015 A1
(43) Pub. Date: Jun. 23, 2011

(54) ACCOMMODATING INTRAOCULAR LENS

(75) Inventors: Aleksey Nikolaevich Simonov, Delft (NL); Michiel Christiaan Rombach, Breda (NL)

(73) Assignee: AKKOLENS INTERNATIONAL B.V., Breda (NL)

(21) Appl. No.: 13/000,145

(22) PCT Filed: Jun. 18, 2009
(86) PCT No.: PCT/NL09/50355

§ 371 Date: Feb. 21, 2011

(30) Foreign Application Priority Data
Jun. 19, 2008 (NL) 2001701

(51) Int. Cl. A61F 2/16

(52) U.S. Cl. 623/6.43; 623/6.56

(57) ABSTRACT

An accommodating intraocular lens (AIOL) construction having optics (2) and haptic (1) connected with the optics for positioning the optics within the eye, wherein the introcular lens construction is made of a single material having spatially-distributed different elasto-mechanical properties, and the elasto-mechanical properties of the haptic differ from the elasto-mechanical properties of the optics. The haptic and the optics made from the same polymer material, i.e., having the same molecular constituency. The optical power of the lens construction can change along with change in the shape of the haptic and intraocular lens construction. The haptic is shaped such that compression along the circumference of the haptic increases the optical strength of the optics.

### PRIORITY CLAIM

This patent application is a U.S. National Phase of International Patent Application No. PCT/NL2009/050355, filed Jun. 18, 2009, which claims priority to Netherlands Patent Application No. 2001701, filed Jun. 19, 2008, the disclosures of which are incorporated herein by reference in their entirety.

### FIELD

The present disclosure relates to an accommodating intraocular lens.

### BACKGROUND

Intraocular lenses (“IOLs”) are generally known to correct refraction of the eye after removal of the natural lens of the eye, as the so-called IOLs for the aphakic eye, with lens removal mostly for treatment of cataracts and, to a lesser extent, for treatment of myopia, as the so-called phakic IOLs, which are, in general, implanted in the anterior chamber of the eye. Standard aphakic monofocal IOLs generally have a fixed optical power and a combination of such lens and progressive spectacles to allow sharp vision at a distance and close-up, for example, reading distance.

Accommodating intraocular lenses (“AIOLs”) allow the eye to focus itself by the natural driving mechanism which also drives the natural lens of the eye. Numerous designs for such accommodating have been proposed, including single optics moving along the optical axis (for example, International Patent Publication No. WO 03/015668), multiple optics moving along the optical axis (for example, International Patent Publication No. WO 2005/104995), multiple optics including cubic surfaces (for example, International Patent Publication Nos. WO 2005/084587 and WO 2006/118452 and Netherlands Patent Application No. 1025622). In addition, there are designs which include flexible optics which change shape and which, in turn, changes the optical properties of the lens. Other designs press pliable material onto a small hole which amplifies the diopter change of the resulting lens (for example, International Patent Publication Nos. WO 2006/040759, WO 2006/103674 and WO 2005/104994).

In “capsular bag refilling designs” (for example, U.S. Patent Publication No. 2001/0049532) the polymer material is supposed to change its shape to vary its optical power due to mechanical forces exerted on the capsular bag of the eye by the natural driving accommodative system. Such capsular bag refilling method does not constitute an IOL/AIOL in the meaning of such IOL/AIOL described in this disclosure and other documents since it constitutes a method, and not a device in itself. The capsular bag refilling material, in itself, is a flexible polymer liquid, does not have haptics/positioning means and the undefined shape and form as a liquid has when not in a molding container, for example, the capsular bag, which shape also defines the shape of the flexible liquid.

Note the terms “pliable”, “elastic”, “flexible” and “elastic/flexible” and their derivatives are used interchangeably in this document, as is the term different “elasto-mechanical properties”. All of these terms refer to the Poisson's ratio of the material. For example, a high elasticity means highly elastic and corresponds to a high Poisson's ratio. Expressed otherwise, a high Poisson's ratio indicates that a contraction as caused by pressure or tension in a first direction of a piece of material leads to an expansion in a direction perpendicular thereto just as the opposite.

### SUMMARY

The present disclosure describes several exemplary embodiments of the present invention.

One aspect of the present disclosure provides an intraocular lens, comprising at least one optical element made of a polymeric material and having variable optical power; and, at least one haptic for positioning the at least one optical element, the at least one haptic being made of a polymeric material having the same molecular constituency as the at least one optical element, each haptic comprising at least two haptic elements, at least two of the haptic elements being positioned on opposite sides of one optical element, wherein the elasto-mechanical properties of the at least one haptic are different than the elasticity of the at least one optical element.

The present disclosure relates to an intraocular artificial lens with variable optical power and comprises optics with variable optical power and positioning means connected with the optics wherein the elasto-mechanical properties of the positioning means differs from the elasto-mechanical properties of the optics. Such deformable optics for the eye are known as prior art and virtually all are made of multiple materials (for example: U.S. Patent Publication Nos. 2007/0021831 and US2005/0085906 and U.S. Pat. No. 5,489,302), generally a rigid material for the haptics and a softer, pliable material for the optics, or even rigid haptics and a near liquid material in an enclosing container with a lens-type shape for the optics. The present disclosure describes a novel concept comprising AIOLs of which the positioning means and the optics are from the same polymer material, i.e., the same molecular constituency. It will be clear that the material should be transparent to be able to function as an optical element or lens. The haptics themselves do not need to be transparent, although the haptics often will be transparent as they are made of the same material as the optical element.

For purposes of the present disclosure, spatially-distributed different elasto-mechanical properties within the same piece of material can be produced at the material producer source, for example, included in a so-called “button”, being a small standard piece of material which is the starting point for the IOL producer, ready for ultra-high precision lathing. Alternatively, the optics and haptics can be manufactured from separate buttons of the same material and different elasto-mechanical properties and the semi-final products subsequently joined by a re-polymerization process including monomers of, again, the same material (see also International Patent Publication No. WO 2006/118452). So, also with re-polymerization, the characteristics of the material will not change and the connection can be regarded as being of the same material as the other components of the IOL/AIOL.

Changing the elasto-mechanical properties of a polymer can be achieved by inter alia changing its water content. For example, well-known hydrophilic acrylate materials, often used for intraocular applications become more elastic by increasing their water contents, from nearly no water (hard/inflexible) to up to 40% water (nearly liquid), and intermediate water contents in a gliding scale of increasing water content and increasing pliability.

Alternatively, such changes in elasticity can also be achieved by varying the degree of polymerization, varying the degree of molecular cross-linking, or varying the degree molecular side-chains. The above methods to vary the degree of elasticity are some examples, and others can likely be applied.

Clearly, multiple areas with different degrees of flexibility/elasticity can be included in the haptics as well as the optics of an intraocular lens construction, and such elasticities can even vary gradually over, for example, an axis of the optics. So, for example, the changing shapes of the optics can be precisely designed and defined as well, and optics with increasing asphericity with increasing optical power can be designed.

The haptics can be in one piece, for example, including the complete rim of the optics, and the design of the AIOL can be such that a change in shape of the haptics will result in a change in the shape of the optics resulting, in turn, in a change in the diopter power of the optics.

Alternatively, the haptics can be constructed of multiple separate pieces and design of the AIOL can be such that a change in shape of the haptics or change in position of the separate pieces relatively to each other or a combination of both effects will result in a change in the shape of the optics resulting, in turn, in a change in the diopter power of the optics.

In all cases, the parts of the haptics adapted to be in contact with the movable part of the natural eye controlling the optical strength of the natural eye are preferably rigid to be able to transfer the movement of the natural eye to the optical element.

Generally, a circumferential compression of the intraocular lens construction should preferably result in an increase in optical diopter power of the optics because such movement is the driving force which also changes the diopter power of the natural lens of the eye. Namely, the ciliary body of the eye of which the ciliary muscle forms a part is positioned just behind the iris and in front of the vitreous body of the eye. In the resting position, the ciliary muscle has a relative large diameter and, when contracting, it contracts to a muscle with a smaller diameter. This muscle drives the accommodative function. The capsular bag is positioned within the ciliary muscle and the natural flexible lens of the eye is positioned in the capsular bag. The capsular bag is connected to the ciliary muscle by zonulea extending substantially radially. The natural accommodation of the eye with a natural lens occurs as follows. During distant viewing, the ciliary muscle is relaxed and has a relatively large diameter. Thus, a pulling force is applied on the zonulae stretching the capsular bag resulting in a relatively flat lens. The natural state of the ciliary muscle results in distant viewing. The ciliary muscle contracts at distant viewing resulting in a smaller diameter. The zonulae relax and the natural lens resumes its natural, more concave shape.

IOLs are of the phakic type (implanted in an eye in which the natural lens remains) or of the aphakic type (implanted as a replacement of the natural lens). The AIOL of the present disclosure can be of a phakic (generally implanted in the anterior chamber of the eye) or an aphakic type.

Most aphakic IOLs/AIOLs are designed to fit the capsular bag of the eye from which the natural lens is removed by the eye surgeon. AIOLs of the present disclosure can be designed to fit the capsular bag and be driven by the ciliary muscle indirectly, and through the action of the zonulae. However, the capsular bag is prone to shrinkage and hardening, which affects the functioning of any AIOL.

Therefore, alternatively, AIOLs of the present disclosure can be designed to fit the sulcus of the eye which positions the AIOLs in front, but outside, the capsular bag. In this position, the AIOL will be driven by the ciliary muscle directly and, in part, by the sulcus itself.

### BRIEF DESCRIPTION OF THE DRAWINGS

Various aspects of the present disclosure are described hereinbelow with reference to the accompanying figures.

FIG. 1 is a cross section view of a first exemplary embodiment in a first, relaxed position;

FIG. 2 is a cross section view of the exemplary embodiment of FIG. 1 in a second, active position;

FIG. 3 is a frontal view of the first exemplary embodiment of FIG. 1;

FIG. 4 is a frontal view of the first exemplary embodiment of FIG. 2;

FIG. 5 is a cross section view of a second exemplary embodiment in a first, relaxed position;

FIG. 6 is a cross section view of a second exemplary embodiment in a second, active position;

FIG. 7 is a cross section view of a third exemplary embodiment of FIG. 5;

FIG. 8 is a cross section view of a third exemplary embodiment of FIG. 6;

FIG. 9 is a cross section view of a fourth exemplary embodiment in a first, relaxed position; and

FIG. 10 is a cross section view of a fourth exemplary embodiment in a second, active position.

### DETAILED DESCRIPTION

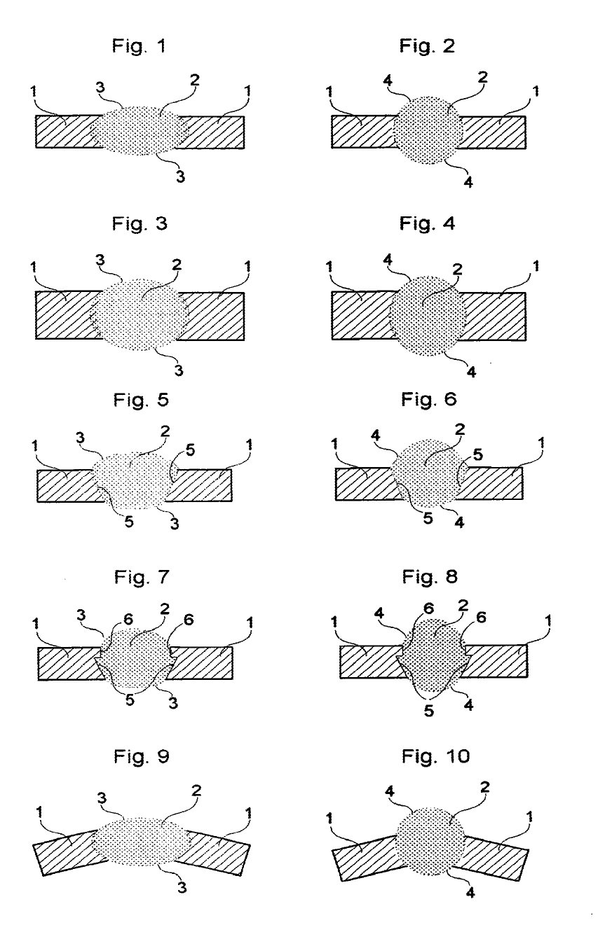

A first exemplary embodiment shown in FIGS. 1-4 discloses an intraocular lens construction comprising an optical element 2 and a haptic 1, the haptic comprising two parts 1a and 1b, located at either side of the optical element 1. The haptic 1 is adapted to locate the intraocular lens construction in the human or animal eye. It is feasible that the haptics may comprise more than two parts, for example, 3-6 elements, in dependence of the location in the eye wherein the intraocular lens construction is fixed. Note that in the drawings the areas with low elasto-mechanical properties are shown in diagonal hash lines and the areas with high elasto-mechanical properties are shown as stippled.

The haptic 1 is made of relatively rigid material, while the optical element is made of relatively soft, pliable, or flexible material, which is at least softer than the material of which the optical element is made. The optical element 2 has a large radius 3 at both sides. This implies that when the optical element 2 is compressed, this compressing will be mainly absorbed by the optical element 2, leading to a change of the shape of the optical element 2 and hence to a change in the optical power of the optical element 2.

A cross section of the optical structure depicted in FIG. 1 in a compressed configuration is depicted in FIG. 2, clearly showing that the optical part has a smaller radius 4 so that its optical power is enlarged.

This also appears in FIG. 4 showing the compressed element depicted in FIG. 2, wherein the distance between the parts 1a, 1b of the haptic 1 is reduced relative to that in FIG. 3. In principle, it is feasible to make use of a single part haptic, but this would require that some parts of the haptic would be relative rigid, while other parts would be relatively flexible, to allow deformation of the optical part.

FIG. 5 shows a second exemplary embodiment mainly in accordance with FIG. 1, but wherein both haptic parts 1a, 1b comprise a funnel-shaped cavity 5 into which the flexible material of the optical part protrudes. The effect thereof is that the radius of the compressed optical part is smaller than that in the first exemplary embodiment, as clearly shown in FIG. 6, leading to an amplification of the lens power.

FIG. 7 shows a third exemplary embodiment which forms a small variation of the second exemplary embodiment, to which a constricting body 6 is added to the haptics 1a, 1b. The shape of the optical element 2 is amended accordingly. The presence of the constricting body 6 further amplifies the effect of the funnel shape so that an even larger variation of the optical power is achieved, as appears in FIG. 8.

A fourth exemplary embodiment is shown in FIGS. 9 and 10. This fourth exemplary embodiment forms again a variation of the first exemplary embodiment, but wherein the haptics 1a, 1b extend at a mutually slightly angled or slanted position to prevent undesired dis-accommodation. Indeed this configuration leads to a slight movement of the optical element 2 in the axial direction which may be used to correct the possibility of the lack of focus due to the change of the optical properties, that is the optical strength of the optical element 2.

Note that the extension of the pliable material can be of a funnel shape protruding in the direction of the optical axis which amplifies the degree of change in shape which, in turn, amplifies the change in diopter value of the resulting lens. A constriction ring can be added to such a funnel design to amplify even more the effects, although the total area of the variable lens will decrease.

In the above-mentioned exemplary embodiments, the shape of the lens perpendicular to the optical axis in the compressed situation is substantially circular. As the compression takes place in only a single direction, this implies that the shape of the lens in the relaxed position is not a circle, but rather an ellipse. Care must be taken to allow sufficient cross section of the optical part so that the full area of retina can be reached by the light. One exemplary construction has optics which are slightly at an angle to the haptics. This is to prevent a possible backward movement of the optics which would result in undesired dis-accommodation.

In the exemplary embodiments disclosed hereinabove, the haptics 1a, 1b are made of rigid material, while the optical element 2 is made of more flexible material. It will be clear that numerous variations may be made to this configuration. It is possible that the extension of the pliable material extends radially from the center of the construction in at least one sector.

It is also possible to use a more gradual change in rigidity, but this may lead to complicated production methods. It seems more logical to use a discrete border between the volumes with different rigidities. Nevertheless, it may be feasible to use more than two different rigidities so that a gradual change of rigidity can be approached more closely.

The exemplary embodiments described hereinabove all relate to a lens construction with the single optical element 2, of which the strength changes due to deformation of the optical element 2. It is, however, also possible to make use of two optical elements 2 cooperating, and wherein the optical power of the elements changes with their mutual position. This can be a movement in the direction of the optical axis or a movement perpendicular to the optical axis. In both cases, the optical elements 2 should be rigid and the flexibility is present in the haptics or positioning elements. It will, however, be clear that the positioning elements will also contain parts with more rigid properties.

For purposes of the present disclosure, an AIOL of the same material as described in this disclosure offers advantages to the material producer as only a single material, albeit in different configurations is used. A further advantage is to a AIOL manufacturer as no combination of different materials is required, just as there is no need for assembly or repolymerization. Yet a further advantage is to doctors and patients, as the single material can desirably be chosen for, inter alia, its biocompatability, and there is no need to prove the biocompatability of combinations of materials and the simple functioning of the device requires only a single element to be implanted into the eye, possibly in the sulcus.

Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention as defined in the following claims.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following inventive concepts.

It should further be noted that any patents, applications and publications referred to herein are incorporated by reference in their entirety.

1. An intraocular lens construction, comprising: optics and positioning means connected with the optics for positioning the optics within an eye,
wherein the optics and the positioning means are made of the same material having spatially-distributed and different elasto-mechanical properties, and
wherein the elasto-mechanical properties of the positioning means differ from the elasto-mechanical properties of the optics.

2. The intraocular lens construction of claim 1, wherein the elasticity is proportionate to the water content of the single material.

3. The intraocular lens construction of claim 1, wherein the elasticity is proportionate to the degree of polymerization of the single material.

4. The intraocular lens construction of claim 1, wherein the elasticity is proportionate to the degree of molecular cross-linking of the single material.

5. The intraocular lens construction of claim 1, wherein the elasto-mechanical properties are determined by molecular side chains of the single material.

6. The intraocular lens construction of claim 1, wherein the positioning means comprises at least two areas having mutually different elasto-mechanical properties.

7. The intraocular lens construction of claim 1, wherein the optics comprise at least two areas having mutually different elasto-mechanical properties.

8. The intraocular lens construction of claim 7, wherein the optics have a gradual change in elasto-mechanical properties along the radius.

9. The intraocular lens construction of claim 1, wherein the optical power changes proportionately with changes in the shape of the positioning means.

10. The intraocular lens construction of claim 1, wherein the optics comprise at least two optical elements and the optical strength of the optics varies with the mutual position of the at least two optical elements.

11. The intraocular lens construction of claim 1, wherein the positioning means are shaped such that compression along the circumference of the positioning means results in an increase in optical strength of the optics.

12. The intraocular lens construction of claim 1, wherein the construction is adapted for implantation in the anterior chamber of the eye.

13. The intraocular lens construction of claim 1, wherein the construction is adapted for implantation in the capsular bag of the eye.

14. The intraocular lens construction of claim 1, wherein the construction is adapted for implantation in the sulcus of the eye.

15. The intraocular lens construction of claim 1, wherein the water content of the single material is up to 40% and wherein the elasticity of the single material is proportionate to the percentage of water content.

16. An intraocular lens, comprising:
(a) at least one optical element made of a polymeric material and having variable optical power; and
(b) at least one haptic for positioning the at least one optical element, the at least one haptic being made of a polymeric material having the same molecular constituency as the at least one optical element, each haptic comprising at least two haptic elements, at least two of the haptic elements being positioned on opposite sides of one optical element,
wherein the elasto-mechanical properties of the at least one haptic are different than the elasto-mechanical properties of the at least one optical element.

17. The intraocular lens of claim 16, wherein each optical element has at least two areas having different elasticity.

18. The intraocular lens of claim 16, wherein each haptic has at least two areas having different elasticity.

19. An intraocular lens construction, comprising: at least one optical element and at least one haptic connected with the at least one optical element for positioning the at least one optical element within an eye,
wherein the at least one optical element and the at least one haptic are made of the same material having spatially-distributed and different elasto-mechanical properties, and
wherein the elasto-mechanical properties of the at least one haptic differ from the elasto-mechanical properties of the at least one optical element.
